(19) United States
(12) Patent Application Publication
(10) Pub. No.: US 2022/0193277 A1
(43) Pub. Date: Jun. 23, 2022

(54) SMART TOILET AND OZONE ASSEMBLY THEREOF

(71) Applicant: CASHIDO CORPORATION, Miaoli County (TW)

(72) Inventors: CHUN-LUNG CHIU, Miaoli County (TW); YUNG-CHIH LEE, Miaoli County (TW)

(21) Appl. No.: 17/130,139

(22) Filed: Dec. 22, 2020

(51) Int. Cl. A61L 2/00; A61L 2/26; C01B 13/11; E03D 9/08; E03D 9/00

(52) U.S. Cl. CPC A61L 2/0088; A61L 2/26; C01B 13/115; E03D 9/08; C01B 2201/12; A61L 2202/11; A61L 2202/15; A61L 2202/17; E03D 9/005

(57) ABSTRACT

A smart toilet and an ozone assembly thereof are provided. The ozone assembly is provided for being connected to a water pipe of a water spraying assembly. The ozone assembly includes an ozone electrolyzer for being assembled to the water pipe and an ozone circuit module that is electrically coupled to the ozone electrolyzer. The ozone electrolyzer is configured to generate an ozone gas to be mixed into the water transported toward the water spraying assembly so as to form ozone water. The ozone electrolyzer is configured to maintain an ozone concentration of the ozone water to be within a range from 0.05 ppm to 0.3 ppm. The ozone circuit module is configured to drive the ozone electrolyzer while the water spraying assembly performs a water drawing process.

### FIELD OF THE DISCLOSURE

The present disclosure relates to a toilet, and more particularly to a smart toilet and an ozone assembly thereof.

### BACKGROUND OF THE DISCLOSURE

Most conventional smart toilets achieve a disinfection function or a deodorization function by releasing negative ions. However, the negative ions have a weak activity and cannot exist for a long time in the conventional smart toilet. In view of this, further improvement can be made to the conventional smart toilet through accurately matching a device having the disinfection function or the deodorization function to the conventional smart toilet.

### SUMMARY OF THE DISCLOSURE

In response to the above-referenced technical inadequacies, the present disclosure provides a smart toilet and an ozone assembly thereof to effectively improve on issues associated with conventional smart toilets.

In one aspect, the present disclosure provides a smart toilet, which includes a toilet seat, a water spraying assembly for cleaning human body, a water tank, a water pipe, a control circuit board, an ozone electrolyzer, and an ozone circuit module. The water spraying assembly is retractably assembled to the toilet seat, and the water spraying assembly is movable relative to the toilet seat between a retracted position and a cleaning position. The water pipe connects the water tank and the water spraying assembly for transporting water in the water tank toward the water spraying assembly. The control circuit board is electrically coupled to the water spraying assembly for controlling the water spraying assembly to be moved to the retracted position or the cleaning position. The control circuit board is configured to drive the water spraying assembly to perform a water drawing process. The water drawing process is performed d by using the water spraying assembly to draw the water in the water tank through the water pipe at a flow rate that is within a range from 0.5 liter/min to 1.5 liter/min. The ozone electrolyzer is assembled to the water pipe. The ozone electrolyzer is configured to generate an ozone gas to be mixed into the water transported toward the water spraying assembly so as to form ozone water, and the ozone electrolyzer is configured to maintain an ozone concentration of the ozone water to be within a range from 0.05 ppm to 0.3 ppm. The ozone circuit module is electrically coupled to the ozone electrolyzer and the control circuit board. The ozone circuit module is configured to drive the ozone electrolyzer while the water spraying assembly performs the water drawing process.

In another aspect, the present disclosure provides an ozone assembly of a smart toilet for being connected to a water pipe of a water spraying assembly. The ozone assembly includes an ozone electrolyzer and an ozone circuit module. The ozone electrolyzer is provided for being assembled to the water pipe. The ozone electrolyzer is configured to generate an ozone gas to be mixed into the water transported toward the water spraying assembly so as to form ozone water. The ozone electrolyzer is configured to maintain an ozone concentration of the ozone water to be within a range from 0.05 ppm to 0.3 ppm. The ozone circuit module is electrically coupled to the ozone electrolyzer, and the ozone circuit module is configured to drive the ozone electrolyzer while the water spraying assembly performs a water drawing process.

Therefore, in the smart toilet or the ozone assembly of the present disclosure, the ozone electrolyzer and the water spraying assembly can be electrically linked to each other through the cooperation of the ozone circuit module and the control circuit board, such that an operation time of the ozone electrolyzer and the water spraying assembly can be precisely assigned.

Moreover, in the smart toilet or the ozone assembly of the present disclosure, the flow rate can be controlled (e.g., 0.5 liter/min to 1.5 liter/min), such that the water drawing process performed by the water spraying assembly can cooperate with the operation of the ozone electrolyzer and precisely generate the ozone water having the ozone concentration within a range from 0.05 ppm to 0.3 ppm.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The present disclosure will become more fully understood from the following detailed description and accompanying drawings.

FIG. 1 is a perspective view of a smart toilet according to a first embodiment of the present disclosure.

FIG. 2 is a planar view showing an arrangement of internal components of the smart toilet of FIG. 1.

FIG. 3 is a functional block diagram of the smart toilet according to the first embodiment of the present disclosure.

FIG. 4 is a perspective view showing an ozone electrolyzer of the smart toilet according to the first embodiment of the present disclosure.

FIG. 5 is an exploded view of the ozone electrolyzer of FIG. 4.

FIG. 6 is an exploded view showing a part of the ozone electrolyzer of FIG. 5.

FIG. 7 is a perspective view of a smart toilet according to a second embodiment of the present disclosure.

FIG. 8 is a planar view showing an arrangement of internal components of the smart toilet of FIG. 7.

FIG. 9 is a functional view of the smart toilet according to the second embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

### First Embodiment

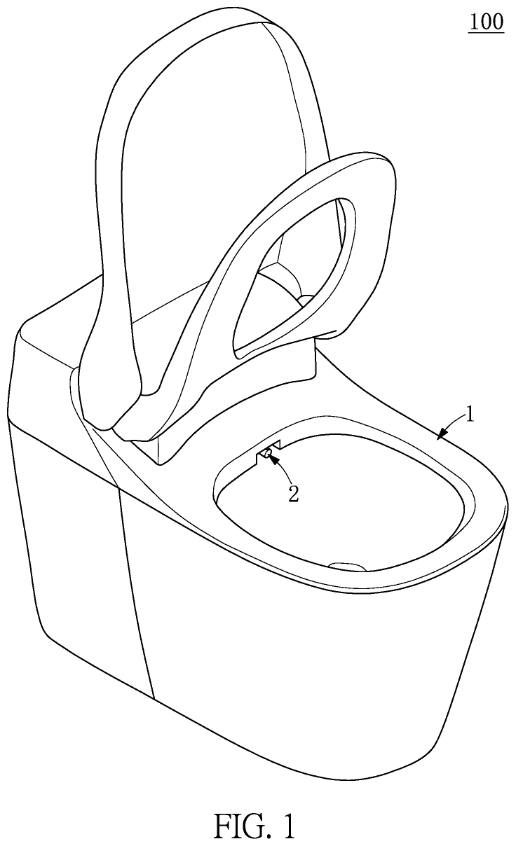

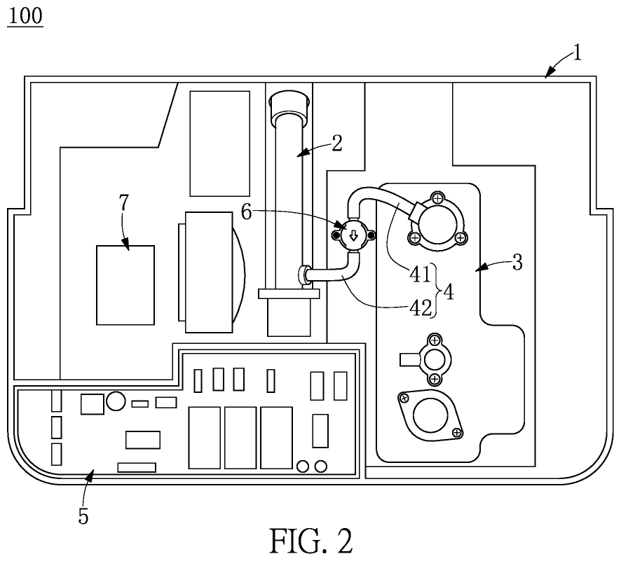

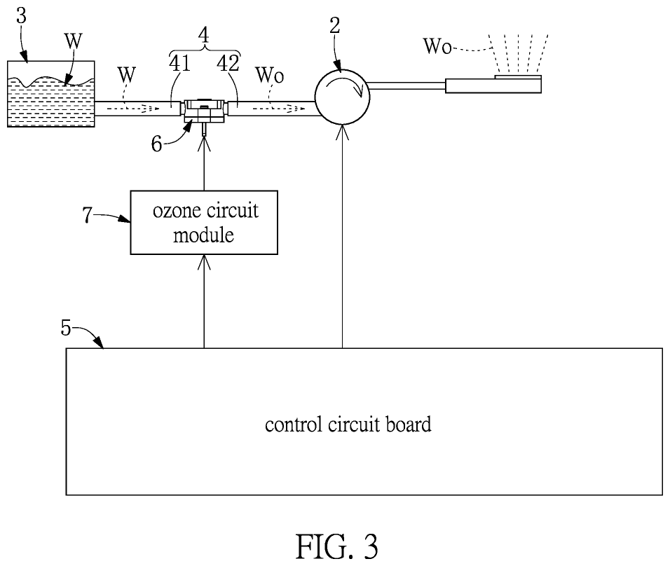

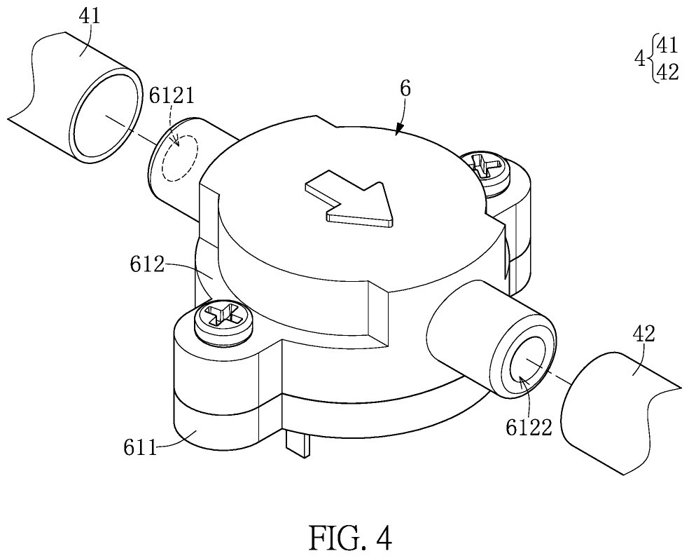

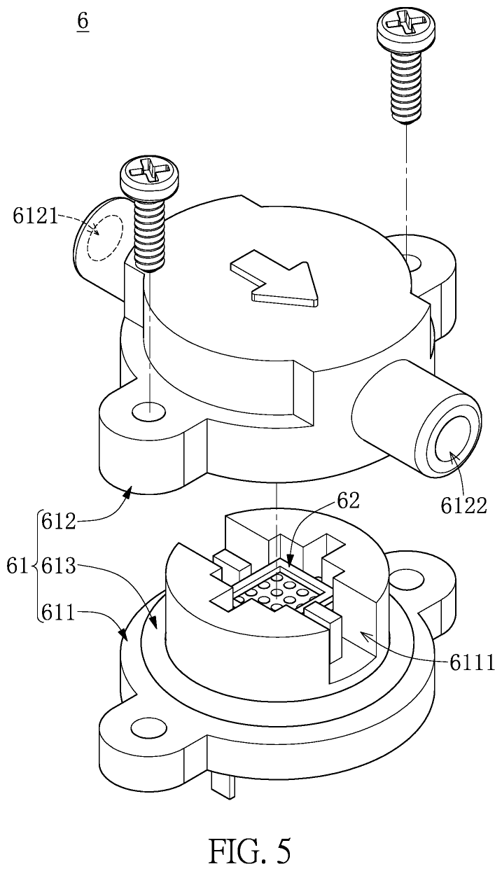

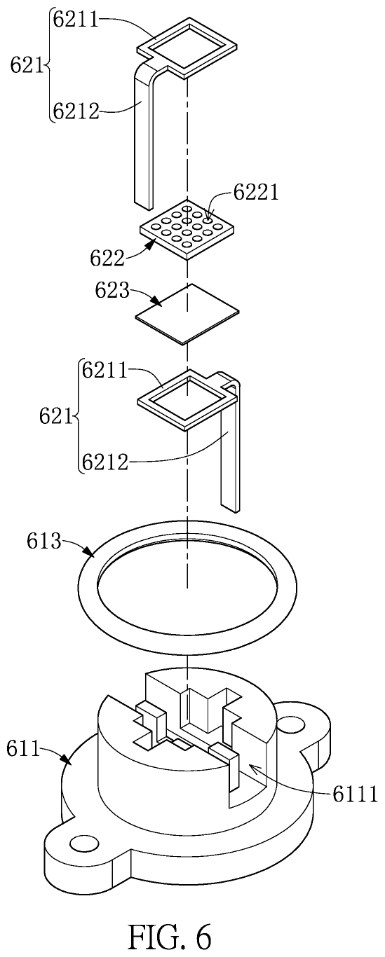

Referring to FIG. 1 to FIG. 6, a first embodiment of the present disclosure provides a smart toilet 100. The smart toilet 100 includes a toilet seat 1, a water spraying assembly 2 retractably assembled to the toilet seat 1 for cleaning a human body, a water tank 3, a water pipe 4 connecting the water tank 3 and the water spraying assembly 2, a control circuit board 5 electrically coupled to the water spraying assembly 2, an ozone electrolyzer 6 assembled to the water pipe 4, and an ozone circuit module 7 that is electrically coupled to the ozone electrolyzer 6 and the control circuit board 5.

It should be noted that the ozone electrolyzer 6 and the ozone circuit module 7 in the present embodiment can be jointly defined as an ozone assembly for being connected to the water pipe 4. Moreover, the ozone assembly in the present embodiment is described in cooperation with the above components (e.g., the toilet seat 1, the water spraying assembly 2, the water tank 3, the water pipe 4, and the control circuit board 5), but the present disclosure is not limited thereto. For example, in other embodiments of the present disclosure (not illustrated), the ozone assembly can be independently used (e.g., sold) or can be used in cooperation with other components.

In addition, the smart toilet 100 (or the ozone assembly) in the present embodiment is limited to generating an ozone gas through the ozone electrolyzer 6, and any device that generates an ozone gas by other mechanisms (e.g., an ultraviolet light mechanism or a high voltage discharge mechanism) is different from the ozone electrolyzer 6 of the present embodiment. The following description firstly describes the components of the smart toilet 100 other than the ozone assembly, and then describes the structure of the ozone assembly structure and the connection between the ozone assembly structure and the other components.

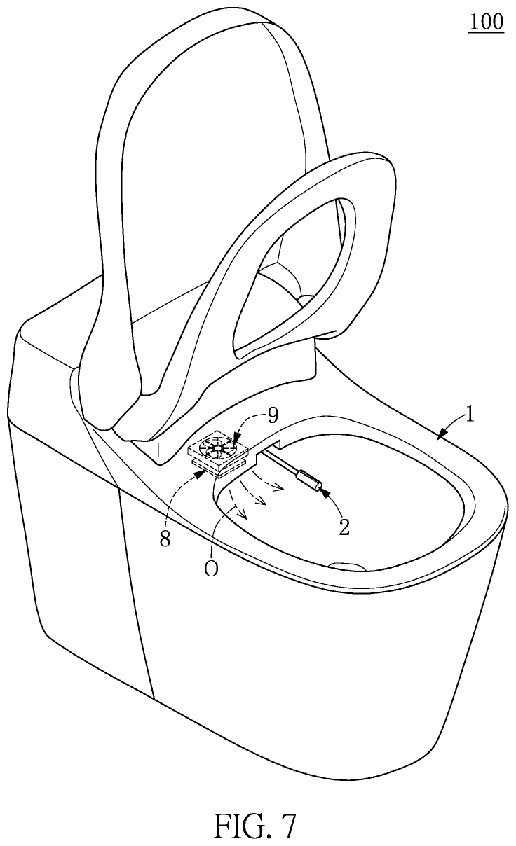

The water spraying assembly 2 is movable relative to the toilet seat 1 between a retracted position (as shown in FIG. 1) and a cleaning position (as shown in FIG. 7). In the present embodiment, the control circuit board 5 can control the water spraying assembly 2 to be moved to the retracted position or the cleaning position by being electrically coupled to the water spraying assembly 2. In addition, in other embodiments of the present disclosure (not illustrated), the control circuit board 5 can be electrically coupled to a manipulation device (e.g., a remote control or a manipulation panel) for a user to operate.

Water W in the water tank 3 can be transported toward the water spraying assembly 2 through the water pipe 4. In the present embodiment, the water pipe 4 includes a first tube 41 and a second tube 42. Two ends of the first tube 41 are respectively connected to the water tank 3 and the ozone electrolyzer 6, and two ends of the second tube 42 are respectively connected to the ozone electrolyzer 6 and the water spraying assembly 2, but the present disclosure is not limited thereto. For example, in other embodiments of the present disclosure (not illustrated), the water pipe 4 can be connected to the water tank 3, the ozone electrolyzer 6, and the water spraying assembly 2 by other structural designs.

Moreover, the control circuit board 5 is configured to drive the water spraying assembly 2 to perform a water drawing process. Specifically, the water drawing process is performed by using the water spraying assembly 2 to draw the water W in the water tank 3 through the water pipe 4 at a flow rate that is within a range from 0.5 liter/min to 1.5 liter/min. In other words, any water drawing process with a flow rate not within the range from 0.5 liter/min to 1.5 liter/min is different from the water drawing process driven by the control circuit board 5 of the present embodiment, and is not suitable for cooperation with the ozone electrolyzer 6 of the present embodiment. Furthermore, the water spraying assembly 2 in the present embodiment includes a nozzle, a motor unit for driving movement of the nozzle, and a water drawing pump that is used for drawing water toward the nozzle, but the present disclosure is not limited thereto.

The ozone electrolyzer 6 is arranged between the first tube 41 and the second tube 42 of the water pipe 4. The ozone electrolyzer 6 is configured to generate an ozone gas O to be mixed into the water W transported toward the water spraying assembly 2 so as to form ozone water Wo. The ozone electrolyzer 6 is configured to maintain an ozone concentration of the ozone water Wo to be within a range from 0.05 ppm to 0.3 ppm. In other words, the cooperation of the ozone electrolyzer 6 and the water drawing process in the present embodiment clearly excludes any ozone gas having an ozone concentration that is not within the range from 0.05 ppm to 0.3 ppm.

Moreover, the ozone circuit module 7 is configured to drive the ozone electrolyzer 6 while the water spraying assembly 2 performs the water drawing process. Specifically, the control circuit board 5 is configured to emit a water drawing signal to drive the water spraying assembly 2 to perform the water drawing process, and the ozone circuit module 7 is configured to receive the water drawing signal and to drive the ozone electrolyzer 6 according to the water drawing signal. In other words, the ozone circuit module 7 can control the operation of the ozone electrolyzer 6 by receiving the water drawing signal of the control circuit board 5.

Accordingly, the ozone electrolyzer 6 and the water spraying assembly 2 in the present embodiment can be electrically linked to each other through the cooperation of the ozone circuit module 7 and the control circuit board 5, such that an operation time of the ozone electrolyzer 6 and the water spraying assembly 2 can be precisely assigned. Moreover, the flow rate can be controlled, such that the water drawing process performed by the water spraying assembly 2 can cooperate with the operation of the ozone electrolyzer 6 and precisely generate the ozone water Wo having the ozone concentration within a range from 0.05 ppm to 0.3 ppm.

Ozone electrolyzers are expensive supplies, and the ozone electrolyzer 6 in the present embodiment can be used effectively and have a longer service life by cooperating with the water drawing process in a more precise manner. In the present embodiment, the water drawing process performed by the water spraying assembly 2 sequentially includes a first spraying period, a second spraying period, and a third spraying period, and the ozone electrolyzer 6 is configured to generate the ozone gas and to maintain the ozone concentration of the ozone water Wo (only) in the second spraying period.

More specifically, the water spraying assembly 2 in the first spraying period is configured to spray the water Win the water tank 3 (e.g., running water) toward a human body, the water spraying assembly 2 in the second spraying period is configured to spray the ozone water Wo toward the human body, and the water spraying assembly 2 in the third spraying period is configured to spray the water W in the water tank 3 (e.g., the running water) toward the human body, but the present disclosure is not limited thereto. For example, in other embodiments of the present disclosure (not illustrated), the water drawing process can be designed to omit the third spraying period, or can be designed to omit the first spraying period and the third spraying period. In other words, the ozone electrolyzer 6 is also capable of further generating the ozone gas and maintaining the ozone concentration of the ozone water Wo in the first spraying period and/or the third spraying period.

It should be noted that a structure having a high use efficiency is provided in the ozone electrolyzer 6 of the present embodiment, but the present disclosure is not limited thereto. In other words, in other embodiments of the present disclosure (not illustrated), the specific structure of the ozone electrolyzer 6 can be changed or adjusted according to design requirements.

In the present embodiment, the ozone electrolyzer 6 includes a housing module 61 and an ozonation module 62 that is assembled in the housing module 61. The housing module 61 has a water flowing space 6111 therein, a water inlet 6121, and a water outlet 6122, the latter two of which being in spatial communication with the water flowing space 6111. The two ends of the first tube 41 are respectively connected to the water tank 3 and the water inlet 6121, and the two ends of the second tube 42 are respectively connected to the water outlet 6122 and the water spraying assembly 2.

Specifically, the housing module 61 in the present embodiment includes a lower case 611, an upper case 512 assembled (e.g., threadedly engaged) to the lower case 611, and a gasket 613 that is assembled to the lower case 611. The water flowing space 6111 in the present embodiment is formed in the lower case 611, and the water inlet 6121 and the water outlet 6122 are respectively formed on two opposite side portions of the upper case 612, but the present disclosure is not limited thereto. The gasket 613 is sandwiched between the upper case 612 and the lower case 611, so that the water flowing space 6111 can be in spatial communication with an external space only through the water inlet 6121 and the water outlet 6122.

Moreover, the ozonation module 62 is assembled in the water flowing space 6111 of the housing module 61. The ozonation module 62 includes two collectors 621 electrically coupled to the ozone circuit module 7 (through at least one cable), at least one electrode pad 622, and a proton exchange membrane (PEM) 623, the latter two of which being arranged between the two collectors 621. In the present embodiment, a total surface area of the at least one electrode pad 622 is within a range from 0.125 cm² to 0.25 cm², and a quantity of the at least one electrode pad 622 is one, but the present disclosure is not limited thereto. For example, in other embodiments of the present disclosure (not illustrated), the quantity of the at least one electrode pad 622 can be two, the PEM 623 is sandwiched between the two electrode pads 622, and the total surface area of the two electrode pads 622 is still within a range from 0.125 cm² to 0.25 cm².

Specifically, any one of the two collectors 621 includes a ring 6211 and a pin 6212 that extends from the ring 6211 to protrude from the housing module 61. Any one of the two collectors 621 is electrically coupled to the ozone circuit module 7 through the pin 6212 thereof, and the at least one electrode pad 622 and the PEM 623 are sandwiched between the rings 6211 of the two collectors 621. Moreover, the at least one electrode pad 622 has a plurality of penetrating holes 6221 corresponding in position to (or in spatial communication with) a space surrounded by each of the rings 6211.

### Second Embodiment

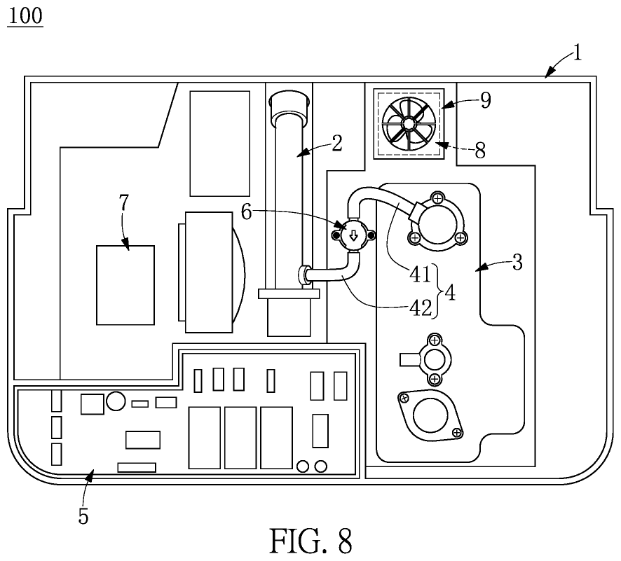

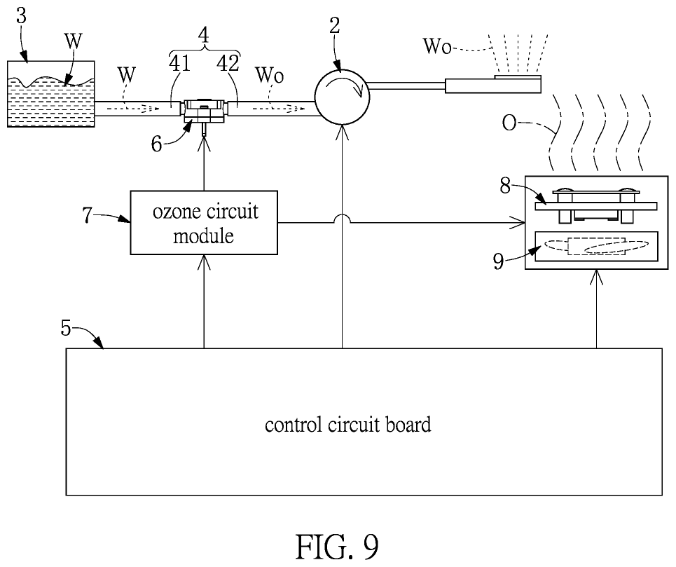

Referring to FIG. 7 to FIG. 9, a second embodiment of the present disclosure is similar to the first embodiment of the present disclosure. For the sake of brevity, descriptions of the same components in the first and second embodiments of the present disclosure will be omitted herein, and the following description focuses on different features between the first and second embodiments. For example, the smart toilet 100 in the present embodiment further includes a direct current (DC) point discharger 8.

Specifically, the DC point discharger 8 is assembled to the toilet seat 1 and is electrically coupled to the ozone circuit module 7. The ozone circuit module 7 is configured to control the operation of the DC point discharger 8 by receiving a signal from the control circuit board 5. For example, before or after the water drawing process is performed by the ozone circuit module 7, the ozone circuit module 7 is configured to drive the DC point discharger 8 to generate an ozone gas O having an ozone concentration that is within a range from 5 mg/hour to 10 mg/hour (after receiving the signal from the control circuit board 5), thereby effectively eliminating any unpleasant odor around the toilet seat 1.

In addition, the smart toilet 100 in the present embodiment further includes a fan 9 assembled to the toilet seat 1 and corresponding in position to the DC point discharger 8. The fan 9 is electrically coupled to the control circuit board 5, so that the control circuit board 5 can be configured to directly control the operation of the fan 9. Accordingly, the fan 9 can blow the ozone gas O generated from the DC point discharger 8 toward the toilet seat 1, thereby rapidly eliminating any unpleasant odor around the toilet seat 1.

In conclusion, in the smart toilet or the ozone assembly of the present disclosure, the ozone electrolyzer and the water spraying assembly can be electrically linked to each other through the cooperation of the ozone circuit module and the control circuit board, such that an operation time of the ozone electrolyzer and the water spraying assembly can be precisely assigned.

Moreover, in the smart toilet or the ozone assembly of the present disclosure, the flow rate can be controlled (e.g., 0.5 liter/min to 1.5 liter/min), such that the water drawing process performed by the water spraying assembly can cooperate with the operation of the ozone electrolyzer and precisely generate the ozone water Wo having the ozone concentration within a range from 0.05 ppm to 0.3 ppm.

In addition, in the smart toilet or the ozone assembly of the present disclosure, before or after the water drawing process is performed by the ozone circuit module, the ozone circuit module is configured to drive the DC point discharger to generate an ozone gas having an ozone concentration that is within a range from 5 mg/hour to 10 mg/hour, thereby effectively eliminating any unpleasant odor around the toilet seat.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A smart toilet, comprising:
a toilet seat;
a water spraying assembly for cleaning a human body, wherein the water spraying assembly is retractably assembled to the toilet seat, and the water spraying assembly is movable relative to the toilet seat between a retracted position and a cleaning position;
a water tank and a water pipe, wherein the water pipe connects the water tank and the water spraying assembly for transporting water in the water tank toward the water spraying assembly;
a control circuit board electrically coupled to the water spraying assembly for controlling the water spraying assembly to be moved to the retracted position or the cleaning position, wherein the control circuit board is configured to drive the water spraying assembly to perform a water drawing process in which the water spraying assembly draws the water in the water tank through the water pipe at a flow rate that is within a range from 0.5 liter/min to 1.5 liter/min;
an ozone electrolyzer assembled to the water pipe, wherein the ozone electrolyzer is configured to generate an ozone gas to be mixed into the water transported toward the water spraying assembly so as to form ozone water, and the ozone electrolyzer is configured to maintain an ozone concentration of the ozone water to be within a range from 0.05 ppm to 0.3 ppm; and
an ozone circuit module electrically coupled to the ozone electrolyzer and the control circuit board, wherein the ozone circuit module is configured to drive the ozone electrolyzer while the water spraying assembly implements the water drawing process.

2. The smart toilet according to claim 1, wherein the water drawing process performed by the water spraying assembly includes a first spraying period and a second spraying period, and the ozone electrolyzer is configured to generate the ozone gas and to maintain the ozone concentration of the ozone water in the second spraying period.

3. The smart toilet according to claim 2, wherein the water drawing process performed by the water spraying assembly includes a third spraying period after the second spraying period, and the ozone electrolyzer is configured to generate the ozone gas and to maintain the ozone concentration of the ozone water only in the second spraying period.

4. The smart toilet according to claim 1, wherein the control circuit board is configured to emit a water drawing signal to drive the water spraying assembly to perform the water drawing process, and the ozone circuit module is configured to receive the water drawing signal and to drive the ozone electrolyzer according to the water drawing signal.

5. The smart toilet according to claim 1, wherein the water pipe includes a first tube and a second tube, and the ozone electrolyzer includes:
a housing module having a water flowing space, a water inlet, and a water outlet, wherein the water inlet and the water outlet are in spatial communication with the water flowing space, two ends of the first tube are respectively connected to the water tank and the water inlet, and two ends of the second tube are respectively connected to the water outlet and the water spraying assembly; and
an ozonation module assembled in the water flowing space of the housing module and including two collectors electrically coupled to the ozone circuit module, at least one electrode pad, and a proton exchange membrane (PEM), wherein the at least one electrode pad and the PEM are arranged between the two collectors, and a total surface area of the at least one electrode pad is within a range from 0.125 cm² to 0.25 cm².

6. The smart toilet according to claim 5, wherein any one of the two collectors includes a ring and a pin that extends from the ring to protrude from the housing module, wherein any one of the two collectors is electrically coupled to the ozone circuit module through the pin thereof, and the at least one electrode pad and the PEM are sandwiched between the rings of the two collectors, and wherein the at least one electrode pad has a plurality of penetrating holes corresponding in position to a space surrounded by each of the rings.

7. The smart toilet according to claim 1, further comprising a direct current (DC) point discharger assembled to the toilet seat and electrically coupled to the ozone circuit module; wherein, before or after the water drawing process is performed, the ozone circuit module is configured to drive the DC point discharger to generate an ozone gas having an ozone concentration that is within a range from 5 mg/hour to 10 mg/hour.

8. An ozone assembly of a smart toilet for being connected to a water pipe of a water spraying assembly, the ozone assembly comprising:
an ozone electrolyzer for being assembled to the water pipe, wherein the ozone electrolyzer is configured to generate an ozone gas to be mixed into water transported toward the water spraying assembly so as to form ozone water, and the ozone electrolyzer is configured to maintain an ozone concentration of the ozone water to be within a range from 0.05 ppm to 0.3 ppm; and
an ozone circuit module electrically coupled to the ozone electrolyzer, wherein the ozone circuit module is configured to drive the ozone electrolyzer while the water spraying assembly performs a water drawing process.

9. The ozone assembly according to claim 8, wherein the ozone electrolyzer includes:
a housing module having a water flowing space, a water inlet, and a water outlet, wherein the water inlet and the water outlet are in spatial communication with the water flowing space, and are configured to be connected to the water pipe; and
an ozonation module assembled in the water flowing space of the housing module and including two collectors electrically coupled to the ozone circuit module, at least one electrode pad, and a proton exchange membrane (PEM), wherein the at least one electrode pad and the PEM are arranged between the two collectors, and a total surface area of the at least one electrode pad is within a range from 0.125 cm² to 0.25 cm².

10. The ozone assembly according to claim 8, further comprising a direct current (DC) point discharger electrically coupled to the ozone circuit module and configured to be assembled to a toilet seat that is installed with the water spraying assembly, wherein the ozone circuit module is configured to drive the DC point discharger to generate an ozone gas having an ozone concentration that is within a range from 5 mg/hour to 10 mg/hour.
